(19) United States
(12) Patent Application Publication
(10) Pub. No.: US 2011/0300239 A1
(43) Pub. Date: Dec. 8, 2011

(54) BLOOD FLOW IMPROVING AGENT

(76) Inventor: Yukio Hasegawa, Osaka (JP)

(21) Appl. No.: 13/201,707

(22) PCT Filed: Feb. 18, 2010
(86) PCT No.: PCT/JP2010/052464

§ 371 Date: Aug. 16, 2011

(30) Foreign Application Priority Data
Feb. 18, 2009 (JP) 2009-035658

(51) Int. Cl. A61K 31/198; A61K 31/195; A61K 31/4172; A61K 31/401; A61K 33/02; A61K 31/37; A61K 33/00; A61P 7/00; A61K 31/197; A61K 31/015; B82Y 5/00

(52) U.S. Cl. 424/719; 514/561; 514/562; 514/567; 514/400; 514/564; 514/423; 514/765; 514/457; 424/724; 977/773

(57) ABSTRACT

An object of the present invention to obtain a blood flow improving agent composed of natural components, which increase activity or circulation of blood flow and accelerate decomposition, detoxication and elimination of toxic substances and supplement each other, and the production method thereof. The blood flow improving agent of the present invention is prepared by blending a nano-colloidized marine organic compound, more than one of trace elements containing minerals extracted from a mineral ore and nano-colloidized plant minerals and amino acids derived from a fuvic acid in nano-bubble water containing dissolved oxygen.

### TECHNICAL FIELD

The present invention relates to a blood flow improving agent and a production method thereof. More specifically, the present invention relates to a blood flow improving agent made from natural components only, into which a marine organic compound, more than one of trace elements and a fulvic acid including plant minerals, whose particle sizes are adjusted to 30 to 50 nm are stably dispersed, and the production method thereof.

### BACKGROUND ART

In recent years, healthy foods and the like produced by extracting a natural component and forming it into the form of beverage and tablets have been attracting attention. Among natural components, minerals or essential trace elements which cannot be obtained from daily diet are especially important and they are known to be necessary for improving lifestyle-related diseases and supplementing insufficient nutriment. However, due to diversification of lives and various individual preferences, desired components and components being deficient or insufficient in the body cannot always be taken appropriately in just proportion, even in the case of taking healthy foods and the like. Therefore, it is impossible to prevent diseases, and one might have an unidentified illness, allergy and other unhealthy conditions which are not said to be illness. However, even in these unidentified illnesses, at least “being deficient in or short of something” will be noted as a common factor of the illnesses. Further, factors for these deficient conditions are not limited to food additives, and materials which DNA does not recognize (such as materials derived from environmental pollution, heavy metals, materials resulting from side effects of pharmaceutical products and the like) are considered to prevent a normal metabolism. Additionally, it has also been found that the blood flow decreases when a normal metabolism is prevented.

### DISCLOSURE OF INVENTION

### Problem to be Solved by the Invention

The present invention is made in view of the above conventional problems, and an object of the present invention is to obtain a blood flow improving agent composed of natural components, which increase an activity or a circulation of the blood flow, accelerate decomposition, detoxication and elimination of toxic substances being left in a body and supplement each other, and a production method thereof. The blood flow improving agent is obtained by blending:

a plant mineral and an amino acid prepared by nano-colloidization of fulvic acid, i.e. the finest electrolyte as a natural material which promotes a catalytic action as a metal-including enzyme or an amino acid and has an action of leading an increase or an activation of cells or an activation of a living body,

essential minerals, essential trace elements and essential ultra-trace elements which are substances extracted from inorganic mineral ores and cannot be obtained from daily diet, substituted by other elements or synthesized in the body, and

a nano-colloidized marine organic compound which is extracted from seaweed and is essential for a hormone balance or a homeostasis function,

into purified water or nano-bubble water containing dissolved oxygen or ozone or nano-bubble water containing dissolved ozone.

### MEANS TO SOLVE THE PROBLEM

The technical background and reason why the components of the present invention are nano-colloidized are that while according to prior arts, a stabilizer, an extender, an antiseptic agent or other stabilizing agents which have nothing to do with the components were used for the stabilization of components and a considerable number of them were those which were chemically synthesized, there has been a necessity for obtaining a high dispersion stability even by effective components only. Therefore, the present invention is intended to find out the interchange point where interparticle attractive force of particles becomes equilibrium while making particle sizes of an organic iodine of a marine organic compound and an fulvic acid gradually smaller, and observing the precipitation or floating of the components at a normal temperature range (approximately 18 to 26° C.).

From the result of the observation, it is recognized that the equilibrium of the interparticle attractive force at about 100 nm is desirable. Then, considering that nano-bubble water is used as a solvent, the particle size is decided to be 30 to 50 nm.

The present invention provides the blood flow improving agent comprising a nano-colloidized marine organic compound in water.

Further, another embodiment of the present invention is the blood flow improving agent further comprising plant minerals and amino acids derived from a nano-colloidized fulvic acid, in which the amino acid is aspartic acid, tricilina, serine, glutamic acid, glycyrin, alanine, cystine, valine, methionine, iwarorine, reucine, tyrosine, phenalene, histidine, lycine, ammonia, allegine, proline, leucine or threonine.

In addition, another embodiment of the present invention is the blood flow improving agent further comprising more than one of trace elements containing minerals extracted from a mineral ore, in which the mineral ore is saprolite of granite.

It is preferable that the above water is nano-bubble water containing dissolved oxygen or ozone.

It is preferable that the size of air bubble in the nano-colloidized marine organic compound is 30 to 50 nm.

It is preferable that the size of air bubble in the nano-colloidized plant minerals and amino acids derived from a fulvic acid dispersed in water is 30 to 50 nm.

It is preferable that the size of air bubble dispersed in the nano-bubble water containing dissolved oxygen or ozone is 30 to 50 nm.

Further, the present invention provides the production method of a blood flow improving agent comprising:

(1) a step of preparing purified water with reverse osmosis membrane equipment,

(2) a step of filling the purified water obtained in the step (1) into a container, and

(3) a step of filling a nano-colloidized marine organic compound into the purified water.

Moreover, another embodiment of the present invention is the production method of a blood flow improving agent comprising:

(1) a step of preparing purified water with reverse osmosis membrane equipment,

(2) a step of filling the purified water obtained in the step (1) into a container, and

(3) a step of filling a nano-colloidized marine organic compound and nano-colloidized plant minerals and amino acids derived from a fulvic acid into the purified water.

Furthermore, another embodiment of the present invention is the production method of a blood flow improving agent comprising:

(1) a step of preparing purified water with reverse osmosis membrane equipment,

(2) a step of filling the purified water obtained in the step (1) into a container, and

(3) a step of filling a nano-colloidized marine organic compound, nano-colloidized plant minerals and amino acids derived from a fulvic acid and trace elements into the purified water.

It is preferable that the step (1) is a step comprising preparing purified water with reverse osmosis membrane equipment and converting it to nano-bubble water containing dissolved oxygen or ozone, and the step (2) is a step comprising filling the nano-bubble water containing dissolved oxygen or ozone into a container after washing the container using the nano-bubble water containing dissolved oxygen or ozone.

### EFFECT OF THE INVENTION

According to the present invention, it is possible to obtain a blood flow improving agent composed of natural components which increase an activity or a circulation of the blood flow, accelerate decomposition, detoxication and elimination of toxic substances being left in a body and supplement each other, and a production method thereof.

Above all, an organic iodine is characterized by two conflicting actions thereof such that it promotes an organic molecule in a toxic substance or a foreign matter to react with a low molecular organic iodine and then to be oxidized, thereby enhancing decomposition, detoxication and elimination of the organic molecule, and that accelerate death of aging cells, phagocytosis of the dead cells in a closed circulation of lymph and generation of new cells under total control of cells. In order to obtain this effect, only the iodine component is extracted at a high purity and colloidized with a particle size being adjusted.

In a fulvic acid being an organic electrolyte, 20 kinds of amino acids contained therein take, through peptide bond, a hormone effect, a neurotransmission effect and an antibacterial effect that are essential to life and are efficient in enhancing the absorption from skin, mouth, mucous, small intestine and the like. The fulvic acid, in conjunction with other polysaccharides, vitamins and enzymes contained therein, has an action of getting one's physical condition in good shape.

### BRIEF DESCRIPTION OF DRAWINGS

FIG. 1 is a flow chart showing the method of the present invention for extracting a nano-colloidized marine organic compound.

FIG. 2 is a flow chart showing the method of the present invention for extracting plant minerals and amino acids derived from a fulvic acid.

FIG. 3 is a flow chart showing the method of the present invention for extracting more than one of trace elements containing minerals extracted from a mineral ore.

FIG. 4 is a flow chart showing the production method of a blood flow improving agent according to one embodiment (Example 1) of the blood flow improving agent of the present invention.

FIG. 5 is a flow chart showing the production method of a blood flow improving agent according to one embodiment (Example 3) of the blood flow improving agent of the present invention.

FIG. 6 is a flow chart showing the production method of a blood flow improving agent according to one embodiment (Example 5) of the blood flow improving agent of the present invention.

### MODE FOR CARRYING OUT THE INVENTION

The present invention relates to the blood flow improving agent prepared by blending a nano-colloidized marine organic compound in water.

Though water is not limited specifically, purified water or the after-mentioned nano-bubble water containing dissolved oxygen or ozone is preferable.

A nano-colloidized marine organic compound means an organic compound which is extracted from seaweed such as nemacystis decipiens, ecklonia cava, laminaria and the like and nano-colloidized to make its particle size in the range of 30 to 50 nm. A brownish-red nonviscous liquid having ph of 5 to 6, degree of transparency of 1 to 2 cm, total iodo content of 70 to 80 ppm, heavy metal content of 10 ppm or less, arsenic content of 10 ppm or less, general microorganisms colony count of 1,000/ml or less, yeasts colony count of 100/ml or less, molds colony count of 100/ml or less and oxidation-reduction potential of approximately 220/mlv or less can be exemplified as a component composing the marine organic compound. When an organic iodine from the marine organic compound, by using the marine organic compound in the above range, is in the optimal range at the existing amount in vivo, the organic iodine may protect thyroid gland from a radioiodine in the air and foods and may prevent an internal cell disruption in advance. Further, these are known as an organic compound having an antitumor action and a healthy growth action. Generally, in the initial stages of extraction of the marine organic compound, the particle size of the marine organic compound is approximately from 100 nm to 1,000 μm and thus the range of the particle size is very wide and the action and effect cannot be brought out.

The blended amount of the marine organic compound is preferably 0.001 to 0.005% per a liter of the above-mentioned water or nano-bubble water. When the blended amount is less than 0.001%, the amount of organic iodine to be present in the body does not reach the optimal range, and when exceeding 0.005%, the organic iodine is excreted if the optimal amount already exists in thyroid gland. In addition, in the case where the particle size of the marine organic compound is beyond the range of 30 to 50 nm, there is a problem that the dispersion stability decreases.

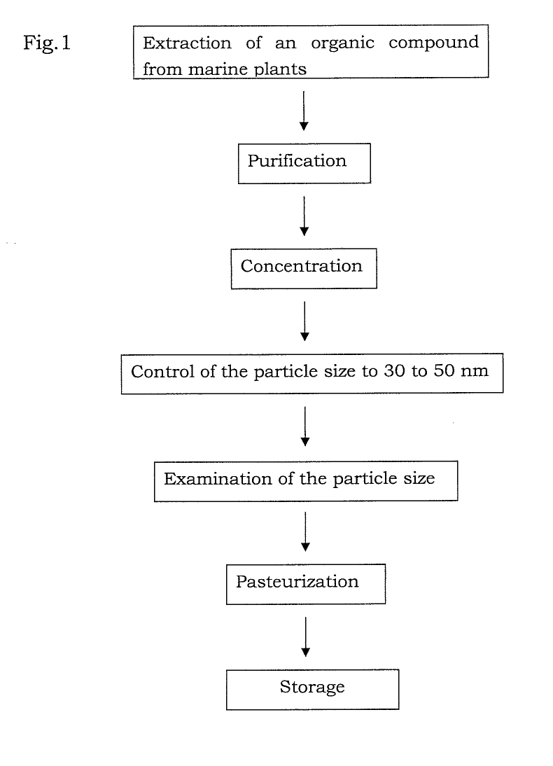

The nano-colloidized marine organic compound is prepared and stored, as shown in FIG. 1, via a step of extracting the organic compound from seaweed as a starting material, a step of purifying the extract, a step of concentrating the purified product, a step of controlling the particle size to 30 to 50 nm, a step of examining the particle size and a step of pasteurizing the resultant product. Though the extraction method is not limited particularly, a supercritical extraction using a supercritical grain extracting device (for example, one manufactured by Mitsubishi Kakoki Kaisha, Ltd.) is preferable from the view point of an easy separation and recovery of an intended material. Further, though the method of controlling the particle size to 30 to 50 nm is not limited particularly, for example, a micro-nanobubble generating device (manufactured by Kyowakisetsu Co., Ltd.) can be used. In addition, the particle size can be affirmed by a zeta potential. The sterilizing temperature is as low as 100° C. or less, preferably 42 to 80° C., more preferably 60 to 63° C. The sterilizing time is preferably 30 to 60 minutes, more preferably 30 minutes or less. It is preferable that the storing temperature is 0 to 3° C.

Nano-colloidization means the state where approximately 5,000 air bubbles exist in 1 mm³.

According to a publication of Advanced Industrial Science and Technology, it was studied that a physiologically active effect of nano-bubble water is derived from its high density aggregability, which is efficient in activating effective bacteria or microorganisms existing in vivo, activating the aerobic blood flow and delivering a necessary component to all over the blood capillary. Moreover, according to the above publication and the research by Tokyo Medical and Dental University, a possibility is discussed that in nano-bubble water containing dissolved ozone, generally air bubbles are crushed in water without rising to the water surface and high temperature and high pressure instantaneously radiated by the crush generate free radical, thereby killing bacteria and viruses. As clear from this, nano-bubble water containing dissolved oxygen and nano-bubble water containing dissolved ozone can be said to be extremely high-safety water without remaining in the body, differently from chemical components.

Moreover, another embodiment of the present invention is the blood flow improving agent further comprising nano-colloidized plant minerals and amino acids derived from a fulvic acid, wherein the amino acid is aspartic acid, tricilina, serine, glutamic acid, glycyrin, alanine, cystine, valine, methionine, iwarorine, reucine, tyrosine, phenalene, histidine, lycine, ammonia, allegine, proline, leucine or threonine.

Examples of the amino acid from fulvic acid is aspartic acid, tricilina, serine, glutamic acid, glycyrin, alanine, cystine, valine, methionine, iwarorine, reucine, tyrosine, phenalene, histidine, lycine, ammonia, allegine, proline, leucine or threonine. Further, minerals are the same as ones exemplified as trace elements except include a fulvic acid iron and a fulvate.

Here, a fulvic acid, which is the finest organic electrolyte as a natural composition and has an action of leading an activation of a living body, has a chelating action or a scavenger action absorbing or eliminating a heavy metal and the like, equiponderates a material allowed to contact therewith and transmits a current as energy for biological properties, as well as being a free radical scavenger (a cleaning agent) and an antioxidant which keep and promote electrochemical equilibrium. Depending on the equilibrium to be needed in cells, the fulvic acid becomes some times a donor and other times an acceptor of an electron. One of induced reactions is an oxidation reaction in which a chemical material provides an electron as a donor and looses it, and the other is a reduction reaction in which an active material receives an electron as an acceptor. At this point, it is reported in many publications that many kinds of trace elements function as an electrode. The fulvic acid which detoxifies contaminants remaining in the body such as an agrichemical, an additive, a dioxin, a heavy metal and the like has an action of adsorbing the chemical compounds from the environment irrespective of the amount or concentration thereof being already dangerous or still not dangerous to a living body. In these years, the fulvic acid having such property has been rapidly researched, as a result, revealed that it has utility in a food chain. Though the full disclosure of a fulvic acid has not been led yet, it has been proved in a large number of researches that the fulvic acid is useful not only for a human body but also a plant. When conducting a germination test of a bean sprout as an example, a clear difference in growth can be observed.

That is, when pouring tap water, mineral water and the blood flow improving agent of the present invention (1,500 times dilution) into each container and putting a seed of bean sprout on cotton therein to observe the condition of the germination, the germinated bean sprout using the blood flow improving agent of the present invention has proved a twice faster growth (length) compared with the growth in the cases of other water.

The blended amount of minerals and amino acids is preferably 0.005 to 0.009% per a liter of the above-mentioned water or nano-bubble water, more preferably 0.005%. When the blended amount is less than 0.004%, there is a problem that the amount does not reach the optimal range for existence in the body, and when the amount exceeds 0.01%, there is a problem that the amount exceeding the optimal range for existence in the body is eliminated.

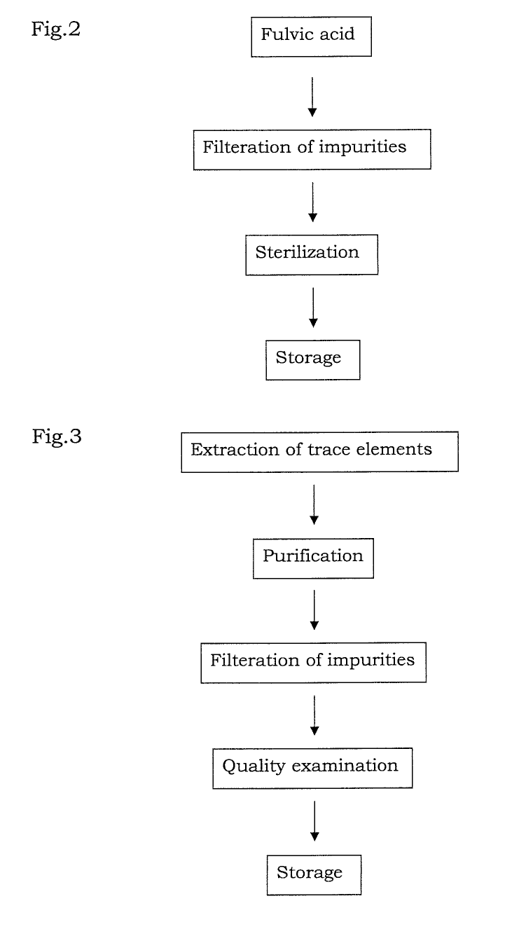

The plant minerals and amino acids derived from a fulvic acid are, as shown in FIG. 2, prepared and stored via a step of filtering impurities from the fulvic acid and a step of sterilizing the filtrate. Though the method of filtering impurities is not limited, generally, a filtering is carried out by using a filter of 1,000 μm, 5,000 μm and 10,000 μm in order being smaller.

The sterilizing temperature is preferably 60 to 63° C., and the sterilizing time is preferably 30 minutes.

In the case of sterilization with a high temperature, there is a problem that a protein structure of a part of amino acids except minerals breaks up and a physiological activity is lost. It is preferable that the storing temperature is 0 to 3° C.

Furthermore, another embodiment of the present invention is the blood flow improving agent further comprising more than one of trace elements containing minerals extracted from a mineral ore, in which the mineral ore is saprolite of granite.

As to the trace elements containing minerals extracted from a mineral ore, examples of the trace elements are calcium, phosphorous, magnesium, potassium, sodium, selenium, silicon, germanium, zinc, manganese, iron, copper, cobalt, nickel, molybdenum, lithium, vanadium, tungsten, barium, titanium, aluminum, strontium, fluorine and the like. Though the mineral ore is not limited specifically, generally saprolite of granite is preferable. The blended amount of the trace elements is preferably 0.001 to 0.007% per a liter of the above-mentioned water or nano-bubble water, and more preferably 0.005%. When the blended amount is less than 0.001%, the amount does not reach the optimal range for existence in the body and when the amount exceeds 0.008%, the amount exceeding the optimal range needed for existence in the body is eliminated.

The trace elements containing minerals extracted from a mineral ore are, as described in FIG. 3, purified and obtained in such a way that the trace elements stably contain the above components.

It is preferable that the air bubble size in the above-mentioned nano-colloidized marine organic compound is 30 to 50 nm.

It is preferable that the air bubbles in the nano-colloidized plant minerals and the amino acids derived from a fulvic acid are dispersed in water at the air bubble size of 30 to 50 nm.

It is preferable that the air bubbles in the nano-bubble water containing dissolved oxygen or ozone are dispersed in water at the air bubble size of 30 to 50 nm.

In the present invention, the nano-bubble water containing dissolved oxygen and the nano-bubble water containing dissolved ozone are solvents for blending a marine organic compound, more than one of trace elements and plant minerals and amino acids derived from a fulvic acid. The air bubble size of the nano-bubble water containing dissolved oxygen and the nano-bubble water containing dissolved ozone is 30 to 50 nm, and since the surface of the air bubbles is negatively charged and the surface area is large, the water is in the state of nano-bubble. The concentration of the dissolved oxygen or ozone is preferably in the range of 80 to 120 ppm. The reason why the air bubble size is decided to the above range is that: not to use stabilizer or antiseptic agent of a chemical component which is generally used, when the size departs from this range, for highly stably dispersing the components and, to obtain an activity by circulation or absorption of the bubbles in the body.

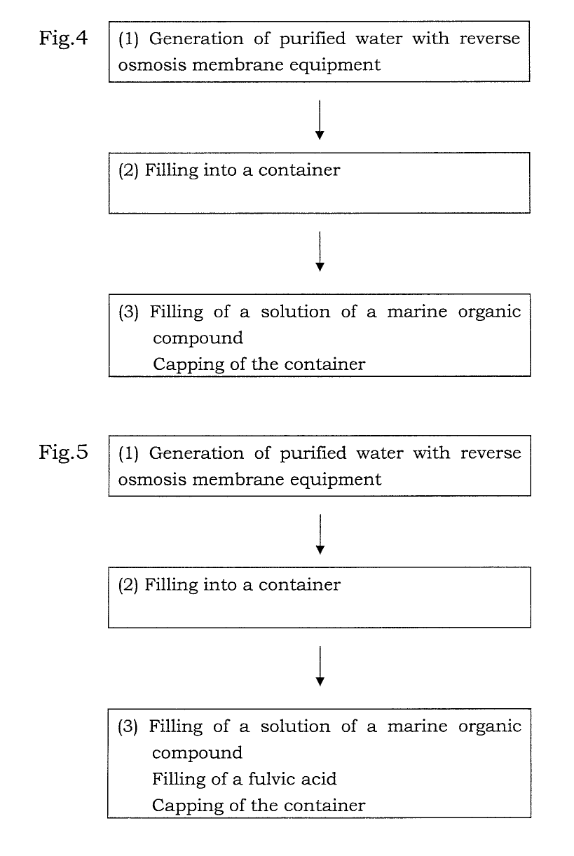

Next, a production method of the blood flow improving agent of the present invention will be explained. As shown in FIG. 4, the blood flow improving agent of the present invention can be produced via steps of: (1) a step of preparing purified water with reverse osmosis membrane equipment, (2) a step of filling the purified water obtained in the step (1) into a container, and (3) a step of filling 2 to 5 ml solution of a marine organic compound per 1,000 ml of purified water, and then capping the container.

Further, another production method of the blood flow improving agent of the present invention will be explained. As shown in FIG. 5, the blood flow improving agent of the present invention can be produced via steps of: (1) a step of preparing purified water with reverse osmosis membrane equipment, (2) a step of filling the purified water obtained in the step (1) into a container, (3) a step of filling 2 to 5 ml solution of a marine organic compound per 1,000 ml of purified water and 5 to 9 ml of plant minerals and amino acids derived from a fulvic acid per 1,000 ml of purified water, and then capping the container.

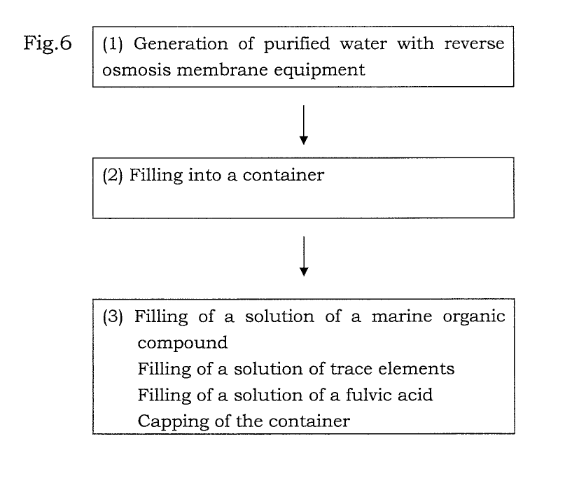

Further, yet another production method of the blood flow improving agent of the present invention will be explained. As shown in FIG. 6, the blood flow improving agent of the present invention can be produced via steps of: (1) a step of preparing purified water with osmosis membrane equipment, (2) a step of filling the purified water obtained in the step (1) into a container, (3) a step of filling 2 to 5 ml solution of a marine organic compound per 1,000 ml of purified water, 3 to 7 ml solution of trace elements per 1,000 ml of purified water and 5 to 9 ml of plant minerals and amino acids derived from a fulvic acid per 1,000 ml of purified water, and then capping the container.

In addition, it is also possible that the above step (1) is a step of preparing purified water with reverse osmosis membrane equipment, and then converting it to nano-bubble water containing dissolved oxygen or ozone, and the above step (2) is a step of filling the nano-bubble water containing dissolved oxygen or ozone into a container after washing the container using the nano-bubble water containing dissolved oxygen or ozone. As described above, the step of washing the container using the nano-bubble water is added in order to utilize a sterilizing power of the nano-bubble water and thereby washing the container. As to the preparation of the nano-bubble water, the above-described micro-nanobubble generating device can be used.

### EXAMPLE

The present invention is then explained in more detail by means of Examples, but the present invention is not limited thereto.

### Example 1

(1) After preparing purified water with reverse osmosis membrane equipment, nano-bubble water containing dissolved oxygen was prepared from the purified water using a micro-nanobubble generating device (BUVITAS manufactured by Kyowakisetsu Co., Ltd.). At this point, the proportion of the dissolved oxygen and ozone was 80 to 90 ppm. (2) After washing a container using the prepared nano-bubble water containing dissolved oxygen or ozone, the nano-bubble water was filled into the container. (3) After filling 2 ml solution of a marine organic compound per 1,000 ml of nano-bubble water, the capping of the container was carried out.

### Example 2

Preparation was carried out in the same manner as in Example 1, except that purified water was used instead of the above nano-bubble water and the step of washing the container in the step (2) was omitted.

### Example 3

(1) After preparing purified water with reverse osmosis membrane equipment, nano-bubble water containing dissolved oxygen was prepared from the purified water using a micro-nanobubble generating device (BUVITAS manufactured by Kyowakisetsu Co., Ltd.). At this point, the proportion of the dissolved oxygen and ozone was 80 to 90 ppm. (2) After washing a container using the prepared nano-bubble water containing dissolved oxygen or ozone, the water was filled into the container. (3) After filling 2 ml solution of a marine organic compound per 1,000 ml of nano-bubble water and 5 ml solution of plant minerals and amino acids derived from a fulvic acid per 1,000 ml of the nano-bubble water, the capping of the container was carried out.

### Example 4

Preparation was carried out in the same manner as in Example 3, except that purified water was used instead of the above nano-bubble water and the step of washing the container in the step (2) was omitted.

Example 5

(1) After preparing purified water with reverse osmosis membrane equipment, nano-bubble water containing dissolved oxygen was prepared from the purified water using a micro-nanobubble generating device (BUVITAS manufactured by Kyowakisetsu Co., Ltd.). At this point, the proportion of the dissolved oxygen and ozone was 80 to 90 ppm. (2) After washing a container using the prepared nano-bubble water containing dissolved oxygen or ozone, the water was filled into the container. (3) After filling 2ml solution of a marine organic compound per 1,000 ml of nano-bubble water, 3 ml solution of trace elements per 1,000 ml of the nano-bubble water and 5 ml of plant minerals and amino acids derived from a fulvic acid per 1,000 ml of the nano-bubble water, the capping of the container was carried out.

Example 6

Preparation was carried out in the same manner as in Example 5, except that purified water was used instead of the above nano-bubble water and the step of washing the container in the step (2) was omitted.

### Comparative Example 1

Preparation was carried out in the same manner as in Example 5, except that the capping of the container was carried out without adding the marine organic compound, the trace elements and the plant minerals and the amino acids derived from a fulvic acid in the step (3).

(Measurement of an Increased Value of Blood Flow)

With reference to Examples 1 and 2 and Comparative Example 1

The blood flow activity in blood capillary was evaluated by a laser doppler equipment.

Model DRT-4 manufactured by Moor Instruments Ltd. was used as the laser doppler equipment for the measurement. The condition was such that after measuring the peripheral blood flow amount in the tip of the middle finger of the left hand of a healthy test subject (35 years old, male), a ten times dilution with water of the blood flow improving agent prepared in Examples 1 and 2 and Comparative Example 1 were taken. Then, after a predetermined period was passed, the peripheral blood flow amount was measured in order (an average value of three times measurement per each) and the observation and the evaluation were conducted.

Then, the variation with a lapse of time of the blood flow was observed and evaluated where the effect of the blood flow was sustained while decreasing from the maximum increased value. The results are shown in Table 1. The reference numeral 1 is a graph showing the variation with a lapse of time of the increased value of the blood flow in terms of the blood flow improving agent of Example 1, the reference numeral 2 is a graph showing the variation with a lapse of time of the increased value of the blood flow in terms of the blood flow improving agent of Example 2 and the reference numeral 7 is a graph showing the variation with a lapse of time of the increased value of the blood flow in terms of the blood flow improving agent of Comparative Example 1.

The increased values of the blood flow of Examples 3 and 4 and Comparative Example 1 were also measured in the same manner. The results are shown in Table 2. The reference numeral 3 is a graph showing the variation with a lapse of time of the increased value of the blood flow in terms of the blood flow improving agent of Example 3 and the reference numeral 4 is a graph showing the variation with a lapse of time of the increased value of the blood flow in terms of the blood flow improving agent of Example 4.

The increased values of the blood flow of Examples 5 and 6 and Comparative Example 1 were also measured in the same manner. The results are shown in Table 3. The reference numeral 5 is a graph showing the variation with a lapse of time of the increased value of the blood flow in terms of the blood flow improving agent of Example 5 and the reference numeral 6 is a graph showing the variation with a lapse of time of the increased value of the blood flow in terms of the blood flow improving agent of Example 6.

As shown in Tables 1 to 3, it has been found that the blood flow improving agents of Examples 1 to 6 show a remarkable increased value of the blood flow compared with the case where the blood flow improving agent of Comparative Example 1 is used.

Explanation of Symbols

1 Increased value of the blood flow of the blood flow improving agent of Example 1

2 Increased value of the blood flow of the blood flow improving agent of Example 2

3 Increased value of the blood flow of the blood flow improving agent of Example 3

4 Increased value of the blood flow of the blood flow improving agent of Example 4

5 Increased value of the blood flow of the blood flow improving agent of Example 5

6 Increased value of the blood flow of the blood flow improving agent of Example 6

7 Increased value of the blood flow of the blood flow improving agent of Comparative Example 1

1. A blood flow improving agent comprising a nano-colloidized marine organic compound in water.

2. The blood flow improving agent of claim 1, further comprising nano-colloidized plant minerals and amino acids derived from a fulvic acid, said amino acid being aspartic acid, tricilina, serine, glutamic acid, glycyrin, alanine, cystine, valine, methionine, iwarorine, reucine, tyrosine, phenalene, histidine, lycine, ammonia, allegine, proline, leucine or threonine.

3. The blood flow improving agent of claim 2, further comprising more than one of trace elements containing minerals extracted from a mineral ore, said mineral ore being saprolite of granite.

4. The blood flow improving agent of claim 1, wherein the water is nano-bubble water containing dissolved oxygen or ozone.

5. The blood flow improving agent of claim 1, wherein the size of air bubble in the nano-colloidized marine organic compound is 30 to 50 nm.

6. The blood flow improving agent of claim 2, wherein the size of air bubble in the nano-colloidized plant minerals and amino acids derived from a fulvic acid dispersed in water is 30 to 50 nm.

7. The blood flow improving agent of claim 4, wherein the size of air bubble dispersed in the nano-bubble water containing dissolved oxygen or ozone is 30 to 50 nm.

8. A production method of the blood flow improving agent of claim 1, comprising:
(1) a step of preparing purified water with reverse osmosis membrane equipment,
(2) a step of filling the purified water obtained in the step (1) into a container, and
(3) a step of filling a nano-colloidized marine organic compound into the purified water.

9. A production method of the blood flow improving agent of claim 2, comprising:
(1) a step of preparing purified water with reverse osmosis membrane equipment,
(2) a step of filling the purified water obtained in the step (1) into a container, and
(3) a step of filling a nano-colloidized marine organic compound, nano-colloidized plant minerals and amino acids derived from a fulvic acid into the purified water.

10. A production method of the blood flow improving agent of claim 3, comprising:
(1) a step of preparing purified water with reverse osmosis membrane equipment,
(2) a step of filling the purified water obtained in the step (1) into a container, and
(3) a step of filling a nano-colloidized marine organic compound, nano-colloidized plant minerals and amino acids derived from a fulvic acid, and trace elements into the purified water.

11. The production method of the blood flow improving agent of claim 10, wherein
the step (1) is a step comprising preparing purified water with reverse osmosis membrane equipment and then converting it to nano-bubble water containing dissolved oxygen or ozone, and
the step (2) is a step comprising filling the nano-bubble water containing dissolved oxygen or ozone into a container after washing the container using the nano-bubble water containing dissolved oxygen or ozone.
